(19) United States
(12) Patent Application Publication
(10) Pub. No.: US 2003/0171873 A1
(43) Pub. Date: Sep. 11, 2003

(54) Method and apparatus for grouping proteomic and genomic samples

(76) Inventor: Bruce Hoff, Venice, CA (US)

Correspondence Address: TOPE-MCKAY & ASSOCIATES, 23852 PACIFIC COAST HIGHWAY #311, MALIBU, CA, 90265, US

(21) Appl. No.: 10/091,660

(22) Filed: Mar. 5, 2002

(51) Int. Cl. G06F 19/00; G01N 33/48; G01N 33/50

(52) U.S. Cl. 702/19; 702/20

(57) ABSTRACT

The present invention provides an apparatus, a method, and a computer program product for clustering proteomic and genomic data. The apparatus comprises a computer system including a processor, a memory, an input, and an output coupled. The apparatus further comprises means, modules, or steps, for (a) receiving a set of data; (b) producing a one-dimensional ordering of the data samples,; (c) configuring a dendrogram from the linearly ordered set of data samples; and (d) outputting the one-dimensional ordering of the data samples and the configuration of the dendrogram; whereby the data samples are clustered in order to allow for efficient analysis to be performed thereon.

### BACKGROUND

(1) Technical Field

The present invention relates to the field of bio-informatics, and more particularly to a tool for grouping large numbers of proteomic and genomic observations.

(2) Discussion

The bioinformatics field, which, in a broad sense, includes any use of computers in solving information problems in the life sciences, and more particularly, the creation and use of extensive electronic databases on genomes, proteomes, etc., is currently in a stage of rapid growth.

In particular, much of the analysis of proteomic and genomic information is performed through the use of microarrays. Microarrays provide a means for simultaneously performing thousands of experiments, with multiple microarray tests resulting in many millions of data samples. To-date, hierarchical clustering has been used, e.g. for analyzing multivariate expression data in order to determine groups of genes that behave similarly. Hierarchical clustering is, however, known to be slow for large numbers of genes, dampening its use in an interactive manner. Also, in its standard form, hierarchical clustering uses a great deal of memory, limiting the number of items that can be clustered. More specifically, standard (agglomerative) hierarchical clustering has a cubic computational time complexity—O(n³). Standard, well-known techniques can be used to speed the procedure up to quadratic time—O(n²), as standard hierarchical clustering has a space complexity of O(n²) .

With the increasing ability to obtain larger quantities of data samples, it is increasingly desirable to develop a system for clustering proteomic and genomic data samples to allow for more rapid analysis. This problem is particularly acute for the development of analysis tools intended to operate in an interactive, or real-time manner. It is an object of the present invention to provide such a system.

### SUMMARY

The present invention provides an apparatus, a method, and a computer program product for clustering proteomic and genomic data. The apparatus comprises a computer system including a processor, a memory coupled with the processor, an input coupled with the processor for receiving proteomic and genomic data and for receiving user input, and an output coupled with the processor for outputting the clustered proteomic and genomic data. The apparatus further comprises means in one embodiment and modules in another embodiment, residing in its processor and memory, for (a) receiving a set of data including n data samples, with each data sample having m characteristics; (b) producing a one-dimensional ordering of the data samples, resulting in a linearly ordered set of data samples including n−1 possible split points; (c) configuring a dendrogram from the linearly ordered set of data samples by iteratively splitting the linearly ordered set of data samples into successive subsets and representing each split in the dendrogram until each subset contains one data sample by traversing the linearly ordered set of data samples and assigning a numerical quality value to each of the n−1 possible split points with at least one of the numerical quality values being a best numerical quality value, and then splitting the set of data at at least one split point based on the best numerical quality values; and (d) outputting the one-dimensional ordering of the data samples and the configuration of the dendrogram; whereby the data samples are clustered in order to allow for efficient analysis to be performed thereon.

In a further embodiment, the means for configuring the dendrogram operates by iteratively splitting the linearly ordered set of data samples by using a local quality technique. This technique assigns a numerical quality value to each possible split point, where each split point resides between two adjacent data samples and where the numerical quality value for each split point is representative of the distance between the two adjacent data samples between which the split point resides. The data set is split at the split point having the greatest quality value, so that each successive split of the data set provides two data subsets with each of the subsets including the data samples on a respective side of the split point.

In another embodiment, the means for configuring the dendrogram iteratively splits the linearly ordered set of data samples by using a within-group variance technique. This technique assigns a numerical quality value to each possible split point, where at each possible split point the set of data samples is divided into two sides, with the numerical quality value at each possible split point being the sum of the variances of the data samples on each side of the split point. The splitting of the data samples occurs at the split point with the lowest such within-group variance, resulting in two linearly-ordered data sample subsets.

In a still further embodiment of the present invention, the means for producing the one-dimensional ordering of the data samples is principal component analysis.

In another embodiment of the present invention, the means for producing the one-dimensional ordering of the data samples is a one-dimensional, self-organizing map.

Each of the means discussed above typically corresponds to a software module for performing the function on a computer. In other embodiments, the means or modules may be incorporated onto a computer readable medium to provide a computer program product. Also, the means discussed above also correspond to steps in a method for clustering proteomic and genomic data.

### BRIEF DESCRIPTION OF THE DRAWINGS

The objects, features and advantages of the present invention will be apparent from the following detailed descriptions of the preferred embodiment of the invention in conjunction with reference to the following drawings where:

FIG. 1 is a block diagram depicting the components of a computer system used in the present invention;

FIG. 2 is an illustrative diagram of a computer program product embodying the present invention;

FIG. 3 is a flow diagram depicting the steps in an embodiment of the method of the present invention.

### DETAILED DESCRIPTION

The present invention relates to the field of bio-informatics, and more particularly to a tool for grouping large numbers of proteomic and genomic observations. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In order to provide a working frame of reference, first a glossary of some of the terms used in the description and claims is given as a central resource for the reader. The glossary is intended to provide the reader with a “feel” for various terms as they are used in this disclosure, but is not intended to limit the scope of these terms. Rather, the scope of the terms is intended to be construed with reference to this disclosure as a whole and with respect to the claims below.

Then, a brief introduction is provided in the form of a narrative description of the present invention to give a conceptual understanding prior to developing the specific details.

### (1) Glossary

Before describing the specific details of the present invention, it is useful to provide a centralized location for various terms used herein and in the claims. The terms defined are as follows:

Dendrogram—A graphic scheme for displaying a hierarchy of groupings of items.

Means—The term “means” as used with respect to this invention generally indicates a set of operations to be performed on a computer. Non-limiting examples of “means” include computer program code (source or object code) and “hard-coded” electronics. The “means” may be stored in the memory of a computer or on a computer readable medium.

Principal Component Analysis—A method for taking multivariate data and deriving an axis of projection that maximally preserves the variance of the data.

### (2) Introduction

Data analyzed by microarray experiments are often grouped so that similar data are clustered together. Current approaches using standard hierarchical clustering techniques are slow for large numbers of data samples and also consume a great deal of computer memory, both of which result in systems that are both cumbersome in terms of time, and are inapplicable in an interactive fashion. The present invention overcomes these difficulties by using a clustering technique that has a time complexity of O(n log n), which is much faster than standard agglomerative clustering techniques, especially as the number of clustered items increases. The technique used in conjunction with the present invention is “divisive”, rather than agglomerative, meaning that the items being clustered are successively split into smaller and smaller clusters. The possible divisions of a group of n data samples into two groups number 2ⁿ, yielding a complexity of O(2ⁿ) for clustering, yielding a naïve (or “obvious”) divisive algorithm that is much worse than standard hierarchical clustering. Instead, the present invention uses a heuristic for splitting the two groups which yields a “splitting” process that takes linear time and an overall complexity averaging O(n log n). As a further benefit, the technique determines the configuration of the tree, i.e. a way to draw the dendrogram such that similar samples (e.g. genes) are placed next to each other for display purposes. This result would generally take a great deal of time to compute, but with the present invention, it requires no additional computation.

### (3) Physical Embodiments of the Present Invention

The present invention has three principal “physical” embodiments. The first is an apparatus for plotting proteomic and genomic information, typically in the form of a computer system operating software of in the form of a “hard-coded” instruction set. The second physical embodiment is a method, typically in the form of software, operated using a data processing system (computer). The third principal physical embodiment is a computer program product. The computer program product generally represents computer readable code stored on a computer readable medium such as an optical storage device, e.g., a compact disc (CD) or digital versatile disc (DVD), or a magnetic storage device such as a floppy disk or magnetic tape. Other, non-limiting examples of computer readable media include hard disks and flash-type memories. These embodiments will be described in more detail below.

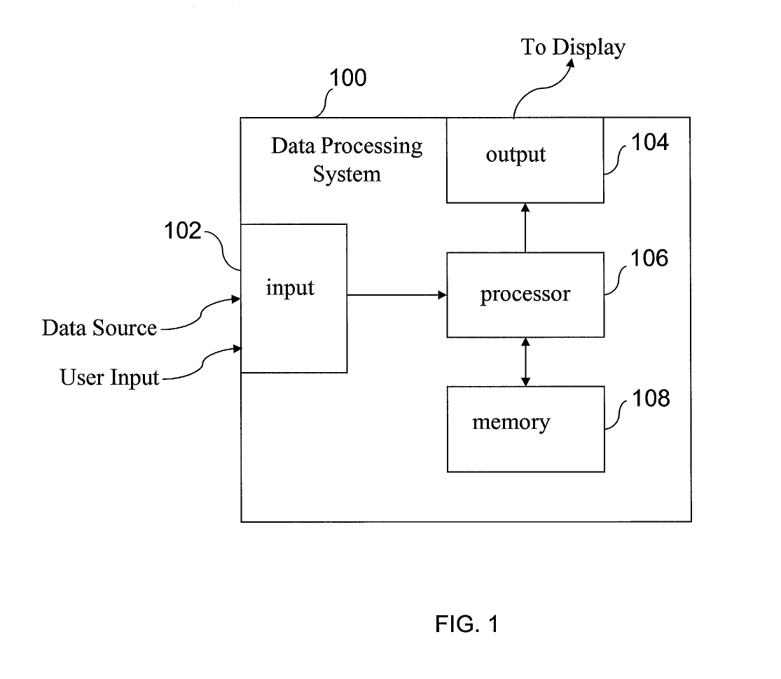

A block diagram depicting the components of a computer system used in the present invention is provided in FIG. 1. The data processing system 100 comprises an input 102 for receiving proteomic and genomic data from a data source and for receiving user input from an input device such as a keyboard. Note that the input 102 may include multiple “ports” for receiving data and user input. Typically, user input is received from traditional input/output devices such as a mouse, trackball, keyboard, light pen, etc., but may also be received from other means such as voice or gesture recognition for example. The output 104 is connected with the processor for providing output. Output to a user is preferably provided on a video display such as a computer screen, but may also be provided via printers or other means. Output may also be provided to other devices or other programs for use therein. The input 102 and the output 104 are both coupled with a processor 106, which may be a general-purpose computer processor or a specialized processor designed specifically for use with the present invention. The processor 106 is coupled with a memory 108 to permit storage of data and software to be manipulated by commands to the processor.

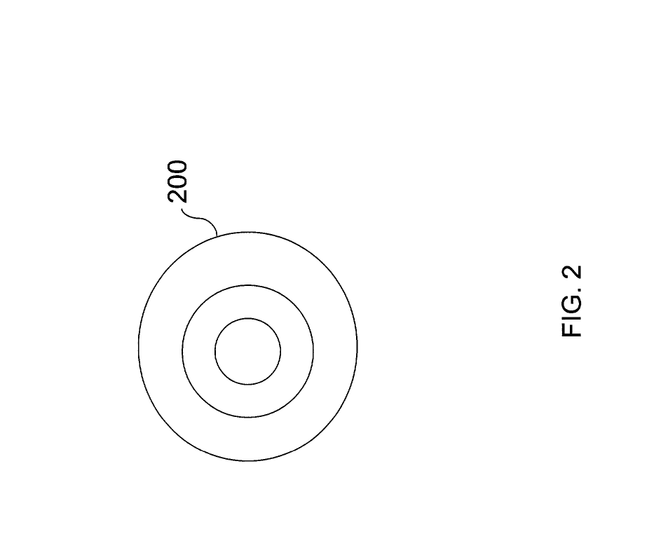

An illustrative diagram of a computer program product embodying the present invention is depicted in FIG. 2. The computer program product 200 is depicted as an optical disk such as a CD or DVD. However, as mentioned previously, the computer program product generally represents computer readable code stored on any compatible computer readable medium.

### (4) The Preferred Embodiments

As stated previously, the present invention provides an apparatus, a method, and a computer program product for efficiently clustering genomic and proteomic data. The present invention uses a one-dimensional self-organizing map in order to perform the search for an optimal splitting point for the data, and produces a faster and less memory intensive system, increasing the size of the largest dataset that can be analyzed within fixed constraints of space and time, thus making the process more interactive, benefiting life scientists.

As mentioned, the technique of the present invention is “divisive” rather than agglomerative, meaning the items (data) being clustered are successively split into smaller and smaller clusters. If the complexity of splitting n items into two groups is x, then the average complexity of the entire process is O(x log n) . If a “brute force” technique was used, then all possible divisions of n items into the two groups would be considered. The possible divisions of a group of n data samples into two groups number 2ⁿ, yielding a complexity of O(2ⁿ), much worse than standard hierarchical clustering. Instead, a heuristic is used for splitting the two groups. First, a one-dimensional self-organizing map is run on the n items, ordering them in a linear fashion as an ordered list. Next, each of the n−1 potential places where the list may be split is considered, and the optimum is selected. Each split-point evaluation requires constant time, using one of the two possible evaluation techniques described below. Thus, this “splitting” process requires O(n) time, and the clustering takes O(n log n) time after computing the one-dimensional self-organizing map. Note that the one-dimensional self-organizing map only need be computed once, before clustering begins, and takes an estimated O(n log n) time, thus the entire process takes O(n log n) time.

As also mentioned before, the present invention also determines the configuration of the hierarchical tree, whereas clustering alone only determines the grouping of the elements. For each grouping, there are many ways to draw the “dendrogram.” It is desirable to draw the dendrogram such that similar elements are near each other, but there are O(2ⁿ) number of configurations to consider, so determining the configuration may be more time consuming than performing the clustering. However, for the technique of the present invention, the one-dimensional self-organizing map determines the ordering of the elements initially, even before the clustering begins. No additional time is required for computing the configuration.

The efficiency provided by the technique of the present invention is important.

For example, if ten thousand (10⁴) genes were clustered, then O(n²) would be on the order of one hundred million (10⁸), while O(n log n) is on the order of only forty thousand (4×10⁴) . As the number of genes to cluster increases, so does the advantage provided by the present invention.

A. One-Dimensional Self-Organizing Map

In a typical example of the use of the present invention in conjunction with genetic information, a list of measurements of n genes is input into a data processing system. Each of the measurements of the n genes includes a list of m measurements (e.g. one measurement for each of the m experiments). A one-dimensional self-organizing map is a technique for adjusting a deformable map to coincide with a given set of data. The map includes a list of 2n nodes connected by arcs in a one-dimensional topology. Each node has an associated m-dimensional vector, placing it in “gene space.” Thus, the whole map is a one-dimensional structure lying in m-dimensional space. The self-organizing map technique gradually moves nodes toward genes in the m-dimensional space. As this occurs, neighboring nodes are moved along. Finally, the one-dimensional structure connects the genes in such a way that the set of genes can be traversed, one at a time, in an order such that successive genes tend to be close in the m-dimensional space. As this process runs, the size of the neighborhood of nodes “dragged along” is reduced; when the neighborhood size reaches zero, it stops. The reduction schedule is such that the number of iterations is logarithmic in the initial neighborhood size which is, in turn, proportional to n. Thus, the number of iterations is O(log n). During each iteration, a node is chosen and the nearest gene is to be found. This process is no worse than the linear process of searching through all genes using brute force. Therefore, the self-organizing map process takes no more than O(n log n) time. The output of the one-dimensional self-organizing map is a linear ordering of the genes.

B. Divisive Clustering

After the self-organizing map process has been completed, divisive clustering begins with the entire gene set. The set is split in two, and then each subset is iteratively split in two until the subsets contain just one gene. When the subsets include just one gene, the process halts. The ordered list of n genes is then traversed, considering each of the n−1 “split points.” At each point, a numerical value is associated with the quality of the split point. A variety of metrics may be used for this purpose, two examples of which are provided herein, both of which take constant time to compute, i.e. the time to compute them is independent of the number of items in the groups. Using one of these metrics, the time to split n ordered items is linear (O(n)). The process proceeds iteratively. As a more specific example, let the n items be split into two groups, one numbering x and the other numbering n-x. The time to split each of these is O(x)+O(n−x)=O(n) . Conceiving the resulting dendrogram, each “level” of the tree requires O(n) to compute. A tree typically has depth of O(log n), so the overall complexity of the technique is O(log n) times O(n) or O(n log n).

C. Splitting Metrics

If the ordered genes are indexed 1, 2, 3, . . . , i, i+1, . . . , n, then a splitting metric computes the quality of splitting the group into (1, 2, . . . i) and (i+1, . . . , n), for all possibilities i=1, 2, . . . , n−1. It does this by assigning a numerical value with each splitting position and the position with the optimal value is then chosen.

i. Local quality technique

If the m-dimensional vector for the ith gene is given by g(i), then the local quality algorithm is distance (g(i), g(i+1)), i.e. the local discontinuity in the gene list. The split point with the largest value is then chosen. Clearly the computation for each split point is independent of the number of genes, n, and is therefore of constant time complexity.

ii. Within group variance

This metric computes the summed squared distance of g(j),j=1 to i, from the mean of g(j),j=1 to i, i.e., the “within-group variance”. This is added to the within-group variance of genes g(i+1) to g(n). The within-group variance value is computed for each split point, and the split point with the smallest value is then chosen. In a naive implementation, the value is computed in linear time for each split point. However, a constant time technique is possible, recognizing that in constant time an update can be computed, transforming the within group variance value at split point i to that at i+1.

It is important to note that the present invention does not generate a matrix of the distances between all genes. Such a matrix is typical in agglomerative clustering, and uses quadratic, or O(n²), memory, creating a tremendous overhead cost when large data sets are analyzed. The technique of the present invention uses only linear, or O(n), memory, storing the original data and related information of the same order of magnitude (e.g. the one-dimensional self-organizing map and the cluster tree each require only linear memory).

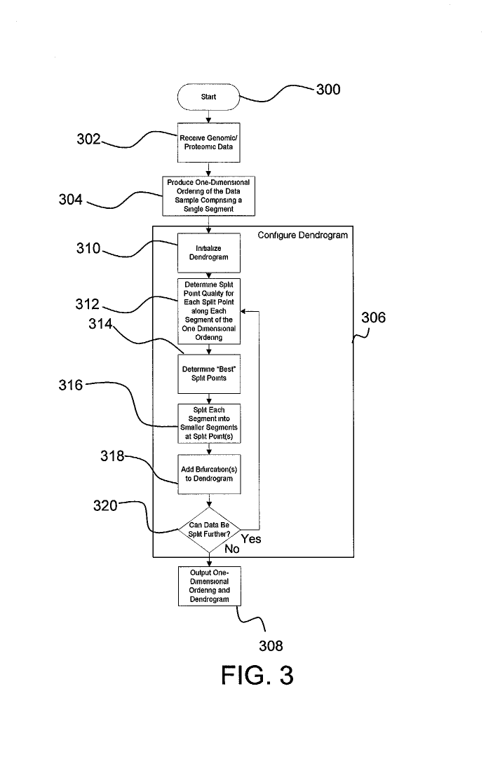

A flow chart depicting the steps of method of the present invention is depicted in FIG. 3. Note that the steps of the flow chart map directly to the “means” in the apparatus and the computer program product embodiments. The flow chart begins with a starting block 300. After the start of the method, genomic and proteomic data is received 302 into the memory of a computer system. In the next step, a one-dimensional ordering of the data is produced in which the data is organized as a single data segment 304 or group. In this step, the data is projected onto a one dimensional line, with each data sample residing on a point along the line. The one-dimensional ordering can be performed, for example by principal component analysis or by the use of a one-dimensional self-organizing map, as discussed in more detail above. After the one-dimensional ordering 304 has occurred, a step of configuring a dendrogram 306 in order to represent the data in a tree-type structure. In the diagram, the step of configuring the dendrogram 306 is depicted as a series of sub-steps 310, 312, 314, 316, 318, and 322. Once the dendrogram has been configured, the one-dimensional ordering and the dendrogram are outputted from the computer system in an outputting step 308.

Referring to the step of configuring the dendrogram 306, first, a single level dendrogram is created in an initializing step 306. After the single level dendrogram has been created, a split point quality is determined for each split point along the set of data 312. Note that the process of generating the dendrogram involves a recursive splitting of the data set into smaller groups, or subsets, at split points, with split points occurring between each pair of adjacent data points. The determination of which split point to use for splitting the data set, or for splitting the subsets at further points in the recursion, is made by assigning a quality value to each split point. The quality value is a measure of the split point's quality for use as a dividing point of the data. A wide variety of criteria may be used for assigning quality values to the split points, two non-limiting examples of which include a local quality technique and a within-group variance technique.

In the local quality technique, a numerical quality value is assigned to each possible split point, where each split point is defined as a point residing between two adjacent data samples. In this case, the numerical quality value for each split point is representative of the distance between the two adjacent data samples between which the split point resides. The data set is split at the split point having the greatest quality value, so that each successive split of the data set provides two data subsets with each of the subsets including the data samples on a respective side of the split point.

In the within-group variance technique, a numerical quality value is assigned to each possible split point, where at each possible split point the set of data samples is divided into two sides, with the numerical quality value at each possible split point being the sum of the variances of the data samples on each side of the split point. The splitting of the set of data samples occurs at the split point with the lowest within-group variance, resulting in two linearly-ordered data sample subsets. More detail regarding both the local quality technique and the within-group variance technique are provided above.

After split point qualities have been assigned to each split point along each segment of the one-dimensional ordering 312, a step of determining the best split point(s) is performed 314. Note that there may be more than one “best” split point for a particular data segment. In cases where this is the case, the segment may be split into more than two subsets. In determining whether more than one “best” split point exists, for example, in a case where a local quality value technique is used, if there are two very similar local quality values (e.g. two data pairs on the same segment whose separation distances are nearly equal, or within a predetermined threshold of each other), both may be used for splitting the data.

After the “best” split points have been determined, the segments are split at the “best” split points into smaller segments 316, and the resulting segments (bifurcations in the case of a single “best” split point in the segment to be split) are added (incorporated into) the dendrogram 318 adding an additional level. If the data can be split further (e.g., there exists a segment with more than one data sample), split point qualities have been assigned to each split point along each remaining segment of the one-dimensional ordering 312, and steps 314, 316, and 318 are repeated again.

Once the data can no longer be split, the dendrogram is configured, and the one-dimensional ordering and the dendrogram are outputted from the computer system in the outputting step 308. The dendrogram and the one-dimensional ordering may be outputted in the form of visual information for display on a computer monitor or for printing, or they may be outputted to other modules for further processing.

What is claimed is:

1. An apparatus for clustering proteomic and genomic data, the apparatus comprising a computer system including a processor, a memory coupled with the processor, an input coupled with the processor for receiving proteomic and genomic data and for receiving user input, and an output coupled with the processor for outputting the clustered proteomic and genomic data, wherein the computer system further comprises means, residing in its processor and memory, for:
a. receiving a set of data including n data samples, with each data sample having m characteristics;
b. producing a one-dimensional ordering of the data samples, resulting in a linearly ordered set of data samples including n−1 possible split points;
c. configuring a dendrogram from the linearly ordered set of data samples by iteratively splitting the linearly ordered set of data samples into successive subsets and representing each split in the dendrogram until each subset contains one data sample by traversing the linearly ordered set of data samples and assigning a numerical quality value to each of the n−1 possible split points with at least one of the numerical quality values being a best numerical quality value, and then splitting the set of data at at least one split point based on the best numerical quality values; and
d. outputting the one-dimensional ordering of the data samples and the configuration of the dendrogram; whereby the data samples are clustered in order to allow for efficient analysis to be performed thereon.

2. An apparatus for clustering proteomic and genomic data, as set forth in claim 1, wherein the means for configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

3. An apparatus for clustering proteomic and genomic data, as set forth in claim 1, wherein the means for configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

4. An apparatus for clustering proteomic and genomic data, as set forth in claim 1, wherein the means for producing the one-dimensional ordering of the data samples is principal component analysis.

5. An apparatus for clustering proteomic and genomic data, as set forth in claim 4, wherein the means for configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

6. An apparatus for clustering proteomic and genomic data, as set forth in claim 4, wherein the means for configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

7. An apparatus for clustering proteomic and genomic data, as set forth in claim 1, wherein the means for producing the one-dimensional ordering of the data samples is a one-dimensional, self-organizing map.

8. An apparatus for clustering proteomic and genomic data, as set forth in claim 7, wherein the means for configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

9. An apparatus for clustering proteomic and genomic data, as set forth in claim 8, wherein the means for configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

10. An apparatus for clustering proteomic and genomic data, the apparatus comprising a computer system including a processor, a memory coupled with the processor, an input coupled with the processor for receiving proteomic and genomic data and for receiving user input, and an output coupled with the processor for outputting the clustered proteomic and genomic data, wherein the computer system further comprises, residing in its processor and memory:
a. a receiving module for receiving a set of data including n data samples, with each data sample having m characteristics;
b. an ordering module for producing a one-dimensional ordering of the data samples, resulting in a linearly ordered set of data samples including n−1 possible split points;
c. a dendrogram module for configuring a dendrogram from the linearly ordered set of data samples by iteratively splitting the linearly ordered set of data samples into successive subsets and representing each split in the dendrogram until each subset contains one data sample by traversing the linearly ordered set of data samples and assigning a numerical quality value to each of the n−1 possible split points with at least one of the numerical quality values being a best numerical quality value, and then splitting the set of data at at least one split point based on the best numerical quality values; and
d. an output module for outputting the one-dimensional ordering of the data samples and the configuration of the dendrogram; whereby the data samples are clustered in order to allow for efficient analysis to be performed thereon.

11. An apparatus for clustering proteomic and genomic data, as set forth in claim 10, wherein the dendrogram module iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

12. An apparatus for clustering proteomic and genomic data, as set forth in claim 10, wherein the dendrogram module iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

13. An apparatus for clustering proteomic and genomic data, as set forth in claim 10, wherein the ordering module is principal component analysis module.

14. An apparatus for clustering proteomic and genomic data, as set forth in claim 13, wherein the dendrogram module iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

15. An apparatus for clustering proteomic and genomic data, as set forth in claim 13, wherein the dendrogram module iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

16. An apparatus for clustering proteomic and genomic data, as set forth in claim 10, wherein the ordering module is a one-dimensional, self-organizing map.

17. An apparatus for clustering proteomic and genomic data, as set forth in claim 16, wherein the dendrogram module iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

18. An apparatus for clustering proteomic and genomic data, as set forth in claim 16, wherein the dendrogram module iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

19. A method for clustering proteomic and genomic data on a computer system including a processor, a memory coupled with the processor, an input coupled with the processor for receiving proteomic and genomic data and for receiving user input, and an output coupled with the processor for outputting the clustered proteomic and genomic data, wherein the method comprises the steps of:
a. receiving a set of data including n data samples, with each data sample having m characteristics;
b. producing a one-dimensional ordering of the data samples, resulting in a linearly ordered set of data samples including n−1 possible split points;
c. configuring a dendrogram from the linearly ordered set of data samples by iteratively splitting the linearly ordered set of data samples into successive subsets and representing each split in the dendrogram until each subset contains one data sample by traversing the linearly ordered set of data samples and assigning a numerical quality value to each of the n−1 possible split points with at least one of the numerical quality values being a best numerical quality value, and then splitting the set of data at at least one split point based on the best numerical quality values; and
d. outputting the one-dimensional ordering of the data samples and the configuration of the dendrogram; whereby the data samples are clustered in order to allow for efficient analysis to be performed thereon.

20. A method for clustering proteomic and genomic data, as set forth in claim 19, wherein the step of configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

21. A method for clustering proteomic and genomic data, as set forth in claim 19, wherein the step of configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

22. A method for clustering proteomic and genomic data, as set forth in claim 19, wherein step of producing the one-dimensional ordering of the data samples is performed by principal component analysis.

23. A method for clustering proteomic and genomic data, as set forth in claim 22, wherein the step of configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

24. A method for clustering proteomic and genomic data, as set forth in claim 22, wherein the step of configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

25. A method for clustering proteomic and genomic data, as set forth in claim 19, wherein the step of producing the one-dimensional ordering of the data samples is performed by a one-dimensional, self-organizing map.

26. A method for clustering proteomic and genomic data, as set forth in claim 25, wherein the step of configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

27. A method for clustering proteomic and genomic data, as set forth in claim 25, wherein the step of configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

28. A computer program product for clustering proteomic and genomic data, the computer program product comprising means, stored on a computer readable medium, for:
a. receiving a set of data including n data samples, with each data sample having m characteristics;
b. producing a one-dimensional ordering of the data samples, resulting in a linearly ordered set of data samples including n−1 possible split points;
c. configuring a dendrogram from the linearly ordered set of data samples by iteratively splitting the linearly ordered set of data samples into successive subsets and representing each split in the dendrogram until each subset contains one data sample by traversing the linearly ordered set of data samples and assigning a numerical quality value to each of the n−1 possible split points with at least one of the numerical quality values being a best numerical quality value, and then splitting the set of data at at least one split point based on the best numerical quality values; and
d. outputting the one-dimensional ordering of the data samples and the configuration of the dendrogram; whereby the data samples are clustered in order to allow for efficient analysis to be performed thereon.

29. A computer program product for clustering proteomic and genomic data, as set forth in claim 28, wherein the means for configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

30. A computer program product for clustering proteomic and genomic data, as set forth in claim 28, wherein the means for configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

31. A computer program product for clustering proteomic and genomic data, as set forth in claim 28, wherein the means for producing the one-dimensional ordering of the data samples is principal component analysis.

32. A computer program product for clustering proteomic and genomic data, as set forth in claim 31, wherein the means for configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

33. A computer program product for clustering proteomic and genomic data, as set forth in claim 31, wherein the means for configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

34. A computer program product for clustering proteomic and genomic data, as set forth in claim 28, wherein the means for producing the one-dimensional ordering of the data samples is a one-dimensional, self-organizing map.

35. A computer program product for clustering proteomic and genomic data, as set forth in claim 34, wherein the means for configuring the dendrogram iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

36. A computer program product for clustering proteomic and genomic data, as set forth in claim 34, wherein the means for configuring the dendrogram iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

37. A computer program product for clustering proteomic and genomic data, the computer program product stored on a computer readable medium, and comprising:
a. a receiving module for receiving a set of data including n data samples, with each data sample having m characteristics;
b. an ordering module for producing a one-dimensional ordering of the data samples, resulting in a linearly ordered set of data samples including n−1 possible split points;
c. a dendrogram module for configuring a dendrogram from the linearly ordered set of data samples by iteratively splitting the linearly ordered set of data samples into successive subsets and representing each split in the dendrogram until each subset contains one data sample by traversing the linearly ordered set of data samples and assigning a numerical quality value to each of the n−1 possible split points with at least one of the numerical quality values being a best numerical quality value, and then splitting the set of data at at least one split point based on the best numerical quality values; and
d. an output module for outputting the one-dimensional ordering of the data samples and the configuration of the dendrogram; whereby the data samples are clustered in order to allow for efficient analysis to be performed thereon.

38. A computer program product for clustering proteomic and genomic data, as set forth in claim 37, wherein the dendrogram module iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

39. A computer program product for clustering proteomic and genomic data, as set forth in claim 37, wherein the dendrogram module iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

40. A computer program product for clustering proteomic and genomic data, as set forth in claim 37, wherein the ordering module is principal component analysis module.

41. A computer program product for clustering proteomic and genomic data, as set forth in claim 40, wherein the dendrogram module iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

42. A computer program product for clustering proteomic and genomic data, as set forth in claim 40, wherein the dendrogram module iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.

43. A computer program product for clustering proteomic and genomic data, as set forth in claim 37, wherein the ordering module is a one-dimensional, self-organizing map.

44. A computer program product for clustering proteomic and genomic data, as set forth in claim 43, wherein the dendrogram module iteratively splits the linearly ordered set of data samples by using a local quality technique, in which a numerical quality value is assigned to each possible split point, where each split point resides between two adjacent data samples and where the numerical quality value for each split point is representative of the distance between the two adjacent data samples between which the split point resides, with the data set being split at the split point having the greatest quality value, so that each successive split of the data set provides two data subsets with each of the subsets including the data samples on a respective side of the split point.

45. A computer program product for clustering proteomic and genomic data, as set forth in claim 43, wherein the dendrogram module iteratively splits the linearly ordered set of data samples by using a within-group variance technique, in which a numerical quality value is assigned to each possible split point, where at each possible split point the set of data samples is divided into two sides, with the numerical quality value at each possible split point being the sum of the variances of the data samples on each side of the split point, and where the splitting of the set of data samples occurs at the split point with the lowest such within-group variance, resulting in two linearly-ordered data sample subsets.
